(19) United States
(12) Patent Application Publication
(10) Pub. No.: US 2006/0127308 A1
(43) Pub. Date: Jun. 15, 2006

(54) Method to improve the efficacy of therapeutic radiolabeled drugs

(76) Inventor: Werner Krause, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/009,518

(22) Filed: Dec. 13, 2004

(51) Int. Cl. A61K 51/00; C07F 5/00

(52) U.S. Cl. 424/1.11; 534/14; 534/15

(57) ABSTRACT

Disclosed are radiolabeled drugs and methods to improve their efficacy by using a radiosensitizer such that the radiosensitizer is either part of the radiolabeled drug by directly attaching the radiosensitizer to the radiolabeled drug or by producing a mixture of the radiolabeled drug and an analogue of the drug with the radiosensitizer attached to the drug instead of the radiolabel.

### FIELD OF THE INVENTION

The present invention relates to radiolabeled drugs and describes a method to improve their efficacy by using a radiosensitizer such that the radiosensitizer is either part of the radiolabeled drug by directly attaching the radiosensitizer to the radiolabeled drug or by producing a mixture of the radiolabeled drug and an analogue of the drug with the radiosensitizer attached to the drug instead of the radiolabel.

### TECHNOLOGY BACKGROUND

Radiolabeled antibodies are valuable diagnostic and therapeutic reagents. They are particularly useful as cancer therapeutics. The administration of a radiolabeled antibody with binding specificity for a tumor-specific antigen, coupled to a radioisotope with a short-range, high-energy radiation, has the potential to deliver a lethal dose of radiation directly to the tumor cell.

An example for a radiolabeled antibody is yttrium-90 labeled Zevalin, which targets the CD20 epitope located on B-cells and which is currently used in the treatment of non-Hodgkin Lymphoma (C. Emmanouilides, Semin Oncol. 2003; 30(4):531-44). The radioisotope, yttrium-90, destroys the cells the antibody is attached to and the cells within the range of its radiation. The radiolytic activity of yttrium-90 has been well described (Salako et al. 1998, J. Nucl. Med. 39: 667; Chakrabarti et al., 1996, J. Nucl. Med. 37: 1384).

Further examples for yttrium-90-labeled antibodies are Theragyn (Hird et al., Br. J. Cancer 1993, 68: 403), which is used for the treatment of ovarian cancer and AngioMab, which comprises the monoclonal antibody BC-1 bound through a linker to yttrium-90 and which is administered for treating solid tumors.

Methods relating to chelator and chelator conjugate synthesis are known in the art (e.g. U.S. Pat. No. 4,831,175, U.S. Pat. No. 5,099,069, U.S. Pat. No. 5,246,692, U.S. Pat. No. 5,286,850, and U.S. Pat. No. 5,124,471).

An example for a radiolabeled antibody using an iodine isotope instead of yttrium-90 is Bexxar, which is labeled with iodine-131. Bexxar also targets the CD20 epitope of B-cells and is used for the treatment of non-Hodgkin Lymphoma (BioDrugs. 2003; 17(4):290-5).

Although these radiolabeled drugs are very effective in their indications, there is room for improvement. It has now been found that their efficacy can be increased by applying the radiosensitizing principle such that either an analogue of the radiolabeled drug with a radiosensitizer moiety instead of the radiolabel-carrying moiety being attached to the drug or that an analogue is synthesized which is identical to the radiolabeled drug except for exchanging the radiolabel-carrying moiety for a radiosensitizing moiety. Radiosensitizers are well known in the field (e.g. EP0316967, US2003166692, US2001051760, U.S. Pat. No. 6,589,981).

Dual-agent compounds that combine the antitumor activity of an active drug such as paclitaxel with the radiosensitizing potential of an additional moiety attached to this drug have been described in WO9640091 and in U.S. Pat. No. 5,780,653. However, these agents still need external radiation, which is unspecific and highly damaging to normal tissue, whereas the present invention utilizes its own radiation source and does not need external radiation. Gd-containing complexes used as radiosensitizers have been described in U.S. Pat. No. 5,457,183 and in US2001051760 where Gd-Texaphyrins or Photofrins are used.

Instead of Gd, other metals with radiosensitizing potential might be utilized such as Co(III) or Fe(III) as described in U.S. Pat. No. 4,727,068.

Radiosensitizers attached to liposomes have been described in WO0045845 wherein a radiosensitizer, e.g. 5-iodo-2′-deoxyuridine is attached to the lipids of the lipisome via a hydrophilic polymer chain.

### SUMMARY OF THE INVENTION

The current invention is related to the improvement of efficacy of radiolabeled drugs by radiosensitization, which is introduced via two possible routes. One route consists in attaching or linking a radiosensitizer moiety to the radiolabeled drug, whereas in the second route the radiolabeled drug is mixed with a drug analogue that contains a radiosensitizer in addition to or instead of the radiolabel.

Thus, in one aspect the invention relates to a method for improving the efficacy of a therapeutic radiolabeled drug comprising either

(i) combining the drug with a radiosensitizer moiety attached to the same molecule or

(ii) co-administering a mixture of a radiolabeled drug and a radiosensitizer provided that the radiolabeled drug and the radiosensitizer have substantially the same targeting characteristics.

In other aspects, the invention relates to such methods

wherein said drug has two moieties linked to it, one moiety containing a radiolabel and the other moiety containing a radiosensitizer; and/or

wherein said drug is a small molecule, preferably labeled with a radioisotope; and/or

wherein said drug is a chelate; and/or

wherein said drug contains a chelate; and/or

wherein said drug is a protein; and/or

wherein said drug is a polymer or biopolymer; and/or

wherein said drug is an antibody or an antibody fragment; and/or

wherein said drug is a DNA or RNA or a fragment thereof; and/or

wherein said drug is a carbohydrate; and/or

wherein said drug is a dendrimeric compound; and/or

wherein said drug is contained in or on a liposome or micelle; and/or

wherein said drug comprises a mixture of a radiolabeled drug and an analogue of this drug functioning as or containing a radiosensitizer provided that the radiolabeled drug and the radiosensitizer have substantially the same targeting characteristics; and/or

wherein said radiolabel is selected from alpha, beta and gamma emitters; and/or

wherein said radiolabel is selected from the group of lanthanides; and/or

wherein said radiolabel is yttrium; and/or

wherein said radiolabel is a radioactive halogen; and/or

wherein said radiolabel is iodine; and/or

wherein said radiosensitizer is or contains gadolinium, iodine or boron; and/or

wherein said radiolabel is attached or linked to the drug by a chelator linked to the drug via a bridge; and/or

wherein said chelator or chelate comprises an EDTA, DTPA, or DOTA moiety; and/or

wherein said linked or unlinked chelator or chelate comprises MX-DTPA, phenyl-DTPA, benzyl-DTPA, or CHX-DTPA; and/or

wherein said radiosensitizer is a triiodobenzene moiety; and/or

wherein said radiosensitizer is a borane or carborane moiety; and/or

wherein said antibody is Zevalin; and/or

comprising loading the chelator or chelate on the antibody with a mixture of a radioactive isotope and gadolinium, cobalt or iron; and/or

comprising loading the chelator or chelate on the antibody with a mixture of yttrium-90 and gadolinium, cobalt or iron; and/or

comprising mixing a drug labeled with a radioactive isotope and a drug analogue labeled with gadolinium, cobalt or iron; and/or

comprising mixing yttrium-90 labeled Zevalin with gadolinium-, cobalt- or iron-labeled Zevalin.

### DETAILED DESCRIPTION OF THE INVENTION

Radiosensitizing so far has been understood as administering a compound that is able to increase the damaging potential of external radiation at the site of a tumor. This means that the radiosensitizer has to reach the tumor site at a concentration that is high enough to act as a radiosensitizer and low enough to exclude adverse reactions and to apply external radiation to exactly this site without damaging normal tissue on its way to the tumor site. Since this goal has not yet been achieved satisfactorily, the use of radiosensitizers in medicine has been very limited.

We have now found a way of circumventing these difficulties. With the new method, external radiation is no longer necessary. Instead, radiation is delivered to the tumor site via administration of a radiolabeled drug which accumulates at the tumor site and which subsequently destroys the tumor cells. By combining this targeted delivery of radiation with a targeted delivery of a radiosensitizer, which is either part of the radiolabeled drug or which is delivered concurrently, before or after administration of the radiolabeled drug, the efficacy of treatment is increased further.

Accordingly, there are two possible routes to increase the efficacy of radiolabeled drugs. The first route can be described as follows. An additional moiety with radiosensitizing potential is attached to a radiolabeled drug without affecting its targeting characteristics. An example for this approach is a monoclonal antibody to which a chelator is attached via a linker. The chelator is able to bind radiolabeled isotopes such as yttrium-90. The antibody is directed to an epitope on tumor cells and carries the radioactive isotope directly to the tumor site where the tumor cells are destroyed by radiation. Normally more than one chelator is attached to an antibody. This means that the chelators can be utilized to bind not only the radioisotope but additionally other metal ions that function as radiosensitizers such as gadolinium, cobalt or iron. The advantage of this approach is that radiation and radiosensitizer are in very close proximity—they are combined in the same molecule—and therefore allow for a high sensitizing yield.

Alternatively, the same type of drug—a monoclonal antibody with a chelator attached to it via a linker—can be loaded either with the radioactive isotope (e.g. yttrium-90) or with the radiosensitizing metal (e.g. gadolinium) and the two drugs, which preferably target the same epitope, can be delivered either as a mixture or subsequently to the patient. Alternatively, two different targeting moieties, antibodies, can be used which localize to the same site, e.g., to different epitopes on the same cell. Both drugs will target the tumor and therefore will be in close proximity to each other on the tumor so that effective radiation and radiosensitization is possible. The proximity might, however, not be as close as in the first example, where radiolabel and radiosensitizer are combined in one molecule and therefore not only co-localize on the tumor but also attach to the very same tumor cell.

Instead of using a chelator for binding a radiosensitizing moiety, other radiosensitizing moieties well known in the art might be used. Examples for other radiosensitizing moieties which might be attached to the drug include iodine atoms or iodine-containing moieties, e.g. triiodobenzene derivatives, or boron atoms or boron-containing moieties such as boranes or carboranes. However, any other radiosensitizing moiety known in the field might be used as well, e.g. platinum-containing moieties, imidazoles or others. Instead of coupling the radiosensitizing moiety directly to the radiolabeled drug, an analogue of the radiolabeled drug might be synthesized such that the radiolabel-containing part is exchanged for a moiety containing the radiosensitizer, i.e. an analogue of the radiolabed drug where a radiosensitizer is in the place of the radiolabel. This means that the antibody of the above-mentioned example would contain a radiosensitizer moiety coupled to it.

However, this principle does not exclusively work with antibodies but also with other carriers such as any biopolymer, polymer, liposome or micelle preparation. Even non-polymeric drugs big enough to carry an additional moiety can be utilized for this principle. For example paclitaxel might be modified such that it contains a chelator coupled to it via a linker. The chelator then could bind a radiolabel, e.g. yttrium-90 and/or a radiosensitizing metal ion such as gadolinium.

Further examples would include chelates themselves that are not coupled to any other drugs but which are drugs on its own. In this case the chelates would bind both the radioisotope and the radiosensitizing metal ion, not necessarily in the same molecule, but in the same solution or in a separate preparation.

Instead of using radioisotopes attached to the carrier drug via chelates, the radioisotopes might be attached to the carrier drug directly for example by radioiodination of an antibody. In this case, the same procedure for preparation might be used to couple non-radioactive iodine to the antibody which then functions as a radiosensitizer. This could be done in the same molecule by simply adding non-radioactive iodine to the radioisotope which is used for radioiodination or by coupling non-radioactive iodine to the antibody. Alternatively, the radiosensitizing potential can be increased by not only coupling single iodine atoms to the drug molecule but iodine carriers such as triiodobenzene derivatives.

The agent(s) can be used in the same doses and in the same regimens as for the non-sensitized agent, but lower doses may also be used as a result of the sensitization. When two molecules are involved, they can be administered simultaneously or sequentially in either order. In the latter case one, e.g., the radiosensitizing agent is administered shortly before the other, e.g., the radioactive drug, e.g., about 15-60 minutes before, longer and shorter times also being possible.

All molecules discussed herein can be prepared conventionally by well known labeling, linking, chelating etc., techniques, e.g., as documented in the cited references and others.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

In the foregoing and in the following examples, all temperatures are set forth uncorrected in degrees Celsius and, all parts and percentages are by weight, unless otherwise indicated.

### EXAMPLES

### Example 1

Radiolabeling of Ibritumomab tiuxetan (Zevalin) with ⁹⁰Y is performed according to the procedure described in WO0052031.

Gd-labeled Zevalin (Gd-Zevalin) is synthesized accordingly using a solution of GdCl₃ instead of YCl₃. Alternative methods for reacting GdCl₃ with a chelator have been described in the literature and persons skilled in the art are familiar with these procedures.

Subsequently, both solutions are injected independently into a patient.

### Example 2

Radiolabeling of Zevalin with ⁹⁰Y and Gd is performed according to the procedure described in example 1.

Subsequently, both solutions are mixed with each other and the mixture is then injected into a patient.

### Example 3

Radiolabeling of Zevalin with ⁹⁰Y and Gd is performed by mixing the solutions of YCl₃ and of GdCl₃ each other and using this mixture for labeling of Zevalin. Optimal binding of Gd and ⁹⁰Y is obtained when both lanthanides are present on an equimolar basis. Since YCl₃ normally is used carrier-free, a non-radioactive Y isotope might be added. Subsequently, the solution is injected into a patient.

### Example 4

Polymers with attached chelates are synthesized as described for example in US2003206865 or in WO03013617. Labeling of these polymers with ⁹⁰Y and Gd is performed according to procedures described in the literature.

### Example 5

Liposomes with radiosensitizers attached to the surface are prepared as described in WO0045845. ⁹⁰Y-DTPA is present during the preparation and is subsequently enclosed within the liposomes, which now contain a radiolabeled drug within the micelles and a radiosensitizer on its surface. U.S. Pat. No. 6,475,515 describes in detail how to prepare liposomes containing chelates.

The entire disclosures of all applications, patents and publications, cited herein and of corresponding U.S. Provisional Application Ser. No. 60/528,473, filed Dec. 11, 2003 is incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. A method for improving the efficacy of the drug yttrium-90 labeled Ibritumomab tiuxetan comprising either
(i) modifying the drug by attaching a radiosensitizing Gd moiety to it, or
(ii) combining the drug yttrium-90 labeled Ibritumomab tiuxetan and Ibritumomab tiuxetan which has a radiosensitizing Gd moiety attached to it to form a mixture.

2. A method of administering yttrium-90 labeled Ibritumomab tiuxetan comprising administering to a patient in need thereof
i) a modified yttrium-90 labeled Ibritumomab tiuxetan to which a radiosensitizing Gd moiety is attached, or
(ii) co-administering either as a mixture or separately the drug yttrium-90 labeled Ibritumomab tiuxetan and Ibritumomab tiuxetan which has a radiosensitizing Gd moiety attached to it.

3. A method according to claim 1, wherein the drug and/or the radiosensitized Ibritumomab tiuxetan is contained in or on a liposome or micelle.

4. A method for improving the efficacy of Ibritumomab tiuxetan or already yttrium-90 labeled Ibritumomab tiuxetan comprising either
(i) a) preparing Gd labeled Ibritumomab tiuxetan comprising labeling Ibritumomab tiuxetan with a radiosensitizing Gd moiety or providing already Gd labeled Ibritumomab tiuxetan, and,
b) preparing yttrium-90 labeled Ibritumomab tiuxetan comprising labeling Ibritumomab tiuxetan with yttrium-90 or providing already yttrium-90 labeled Ibritumomab tiuxetan, and
c) administering to a patient either together as a mixture or separately the Gd labeled Ibritumomab tiuxetan and the yttrium-90 labeled Ibritumomab tiuxetan, or,
(ii) a) preparing Gd and yttrium-90 labeled Ibritumomab tiuxetan comprising labeling Ibritumomab tiuxetan with both a radiosensitizing Gd moiety and yttrium-90, and
b) administering to a patient the Gd and yttrium-90 labeled Ibritumomab tiuxetan.

5. A method for improving the efficacy of a therapeutic radiolabeled drug comprising either
(i) a) combining the drug with a radiosensitizer moiety, such that the radiosensitizer moiety attaches to the drug, and
b) then administering to a patient the drug, or
(ii) a) labeling a carrier that has substantially the same targeting characteristics as the radiolabeled drug with a radiosensitizer moiety, and
b) then administering to a patient the radiolabeled drug and the carrier either together as a mixture or separately.

6. A method of administering a therapeutic radiolabeled drug comprising administering to a patient in need thereof
(i) a modified radiolabeled drug to which a radiosensitizing Gd moiety is attached, or
(ii) co-administering either as a mixture or separately the radiolabeled drug and a carrier that has substantially the same targeting characteristics as the radiolabeled drug which has a radiosensitizing Gd moiety attached to it.

7. A method according to claim 5, wherein the carrier and the radiolabeled drug have as a target in a body of a patient the same epitope, or both the carrier and the radiolabeled drug localize in the same site in the body, or attach to different epitopes on the same cell.

8. A method according to claim 5, wherein the carrier is an antibody, biopolymer, polymer, liposome or micelle preparation or a non-polymeric drug.

9. A method according to claim 5, wherein the drug has at least two moieties linked to it, at least one of which moieties contains a radiolabel and at least one of which moieties contains a radiosensitizer.

10. A method according to claim 5, wherein the drug is a chelate or contains a chelate.

11. A method according to claim 5, wherein the drug is a protein, a polymer or biopolymer, antibody or an antibody fragment, DNA or RNA or a fragment thereof, a carbohydrate, or a dendrimeric compound.

12. A method according to claim 5, wherein the drug comprises a mixture of a radiolabeled drug and an analogue of this drug functions as or contains a radiosensitizer provided that the radiolabeled drug and the radiosensitizer have substantially the same targeting characteristics.

13. A method according to claim 5, wherein the radiolabel is an alpha, beta or gamma emitter.

14. A method according to claim 5, wherein the radiolabel is selected from the group of lanthanides.

15. A method according to claim 5, wherein the radiolabel is yttrium.

16. A method according to claim 5, wherein the radiolabel is a radioactive halogen, or iodine.

17. A method according to claim 5, wherein the radiosensitizer is or contains gadolinium, iodine or boron, or is a triiodobenzene moiety or a borane or carborane moiety.

18. A method according to claim 5, wherein the radiolabel is attached or linked to the drug by a chelator linked to the drug via a bridge.

19. A method according to claim 18, wherein the chelator or chelate comprises an EDTA, DTPA, or DOTA moiety.

20. A method according to claim 5, wherein the drug is linked or unlinked chelator or chelate comprises MX-DTPA, phenyl-DTPA, benzyl-DTPA, or CHX-DTPA.

21. A method according to claim 20, comprising loading the chelator or chelate on an antibody with a mixture of a radioactive isotope and gadolinium, cobalt or iron; and/or comprising loading the chelator or chelate on an antibody with a mixture of yttrium-90 and gadolinium, cobalt or iron.

22. A method according to claim 5, wherein the drug is Ibritumomab tiuxetan.

23. A method according to claim 5, comprising mixing a drug labeled with a radioactive isotope and a drug analogue labeled with gadolinium, cobalt or iron; and/or comprising mixing yttrium-90 labeled Ibritumomab tiuxetan with gadolinium-, cobalt- or iron-labeled Ibritumomab tiuxetan.
